(19) United States
(12) Patent Application Publication
(10) Pub. No.: US 2024/0329055 A1
(43) Pub. Date: Oct. 3, 2024

(54) Nanomembrane Device And Method For Biomarker Sampling

(71) Applicant: University of Rochester, Rochester, NY (US)

(72) Inventors: James Lionel McGrath, Rochester, NY (US); Jonathan David Flax, Rochester, NY (US); Kilean Scott Lucas, Billerica, MA (US); Samuel Norman Walker, Rochester, NY (US)

(73) Assignee: University of Rochester, Rochester, NY (US)

(21) Appl. No.: 18/681,988

(22) PCT Filed: Jul. 31, 2022
(86) PCT No.: PCT/US2022/038984

§ 371 Date: Feb. 7, 2024

Related U.S. Application Data
(60) Provisional application No. 63/230,779, filed on Aug. 8, 2021.

(51) Int. Cl. G01N 33/68; B01L 3/00; G01N 33/543; G01N 33/569

(52) U.S. Cl. CPC G01N 33/6863; B01L 3/502761; G01N 33/54386; G01N 33/56966; B01L 2200/0647; B01L 2300/0654

(57) ABSTRACT

A device and related methods for the detection of biomarkers is disclosed. The device includes a nanoporous membrane having a plurality of pores. The nanoporous membrane is configured to capture extracellular vesicles or other biological material. An assay is used, to determine the level of biomarkers of interest that are contained with the captured extracellular vesicles or other biological material

### STATEMENT REGARDING FEDERALLY SPONSORED RESEARCH OR DEVELOPMENT

This invention was made with government support under IIP-1917902 awarded by National Science Foundation, W81XWH-18-1-0560 awarded by Department of Defense and A1147362 awarded by National Institutes of Health. The government has certain rights in the invention.

### TECHNICAL FIELD

The present invention relates generally to nanomembrane devices and methods, and more particularly to a device and method for the sampling of biomarkers.

### BACKGROUND ART

Nanoporous Silicon Nitride Membranes have a variety of applications including, but not limited to, filtering, capturing or otherwise separating out specific analytes from a fluid such as a biofluid. Such a membrane is described, for example, in United States Patent application publication 2016/0199787 A1 to Striemer et al. and entitled Nanoporous Silicon Nitride Membranes, And Methods For Making And Using Such Membranes, the entire disclosure of which is incorporated herein by reference. Other membranes, devices and methods applicable to the present invention and the various embodiments described, depicted and envisioned herein are disclosed in U.S. Pat. No. 8,518,276 entitled Ultrathin Porous Nanoscale Membranes. Methods of Making, and Uses Thereof to Striemer et al. and 8,501,668 entitled Drug Screening Via Nanopore Silicon Filters to MeGrath et al., the entire disclosures of which are incorporated herein by reference in their entirety.

Nanoporous Silicon Nitride Membranes can be used for the capture and retention of Extracellular Vesicles. Extracellular vesicles are lipid bilayer particles derived from several cellular pathways including exosomes, microvesicles, and apoptotic bodies. Exosomes of 30-100 nm diameter are derived from the endosomal pathway. Microvesicles of 100 nm-1 um diameter are derived from the plasma membrane. Extracellular vesicles can be found in biofluids such as blood, plasma, serum, urine, cerebrospinal fluid, aqueous humor, lymph, breast milk, semen, and conditioned cell culture media, among others.

U.S. patent application Ser. No. 16/476,329 entitled “Device and Method for Isolating Extracellular Vesicles From Biofluids” by Dr. James L. McGrath et al. describes a novel nanomembrane that is used for a variety of applications including, but not limited to, capturing extracellular vesicles from a bodily fluid. The entire disclosure of this application is incorporated herein by reference. While there are emerging uses for extracellular vesicles in medical testing and diagnostics, the capture and use of extracellular vesicles for medical testing and therapeutics as further described herein is novel.

### DISCLOSURE OF THE INVENTION

In accordance with the present invention, there is provided a device for the detection of biomarkers, the device comprising a nanoporous membrane comprising a plurality of pores, the nanoporous membrane configured to capture extracellular vesicles, and an assay to determine the level of biomarkers contained with captured extracellular vesicles.

The foregoing has been provided by way of introduction, and is not intended to limit the scope of the invention as described by this specification, claims and the attached drawings.

### BRIEF DESCRIPTION OF THE DRAWINGS

The invention will be described by reference to the following drawings, in which like numerals refer to like elements, and in which:

FIG. 1 depicts capture of exosomes and subsequent biomarker detection on a tangential flow device of the present invention;

FIG. 2 is a chart depicting typical analyte sizes;

FIG. 3 illustrates the labeling of biomarkers on extracellular vesicles in accordance with the present invention;

FIG. 4 is a graph depicting pressure with respect to time for a nanoporous membrane of the present invention;

FIG. 5 illustrates the labeling of extracellular vesicles in solution;

FIG. 6 illustrates the capture of labeled extracellular vesicles in solution using a nanoporous membrane of the present invention; and

FIG. 7 depicts detection of labeled extracellular vesicles using a fluorescent antibody combination.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and drawings attached hereto.

### BEST MODE FOR CARRYING OUT THE INVENTION

The present invention involves the capture, physical retention and labeling of extracellular vesicles from biofluids and related methods for the detection of biomarkers such as, but not limited to, immune checkpoint proteins. Such devices and methods have wide applicability in the medical field where the detection and measurement of specific biomarkers has utility in a variety of endeavors.

The present invention makes use of nanoporous silicon nitride membranes in a device such as a tangential flow device, wherein the extracellular vesicles are captured by a novel, diffusion-driven, physical sieving mechanism, allowing for subsequent isolation and labeling thereof.

The present invention includes a device for the detection of biomarkers, the device comprising a nanoporous membrane comprising a plurality of pores, the nanoporous membrane configured to capture extracellular vesicles, and an assay to determine the level of biomarkers contained with captured extracellular vesicles.

Nanoporous membranes such as nanoporous Silicon Nitride (SiN) membranes can be part of a monolithic structure or a free-standing membrane. Thus, the nanoporous SiN membrane may be supported by a Si wafer or may be independent of the Si wafer.

The SiN membrane can have a range of pore sizes and porosity. For example, the pores can be from 10 nm to 100 nm, including all values to the nm and ranges therebetween. The pores also can be 10 nm or less or even 1 nm or less. For example, the porosity can be from <1% to 40%, including all integer % values and ranges therebetween. In a particular embodiment, the SiN pore sizes range from approximately 5 nm to 80 nm and the SiN porosity ranges from 1% to 40%. Of course, other pore size and porosity values are possible and these are merely listed as examples. The shape of the pores can be modified. For example, conical pores can be produced by reducing RIE etching time.

The SiN membrane can have a range of thickness. For example, the thickness of the membrane can be from 20 am to 100 am, including all values to the nm and ranges therebetween. Of course, other thickness values are possible and these are merely listed as examples.

In an embodiment, the SiN membrane is at least one layer of a layered structure on a substrate (i.e., part of a monolithic structure). For example, the membrane can be a layer on a silicon wafer. The membrane is at least partially free from contact with the adjacent layer (or substrate).

In another embodiment, the SiN membrane is a free-standing membrane. This membrane can have a range of sizes. For example, the membrane can have an area of up to 100 mm.sup.2 and/or a length of up to 10 mm and a width of up to 10 mm when using a Si wafer for support. However, if the membrane is separated from the Si wafer, then a larger area may be available. For example, free-standing circular membranes with diameters of 4 inches, 6 inches, or 8 inches, which may correspond to the silicon wafer size, can be fabricated.

A membrane occupying an entire Si wafer, which is greater than 100 cm.sup.2, can be produced by embodiments of the “lift-off” process discussed herein. For example, SU-8 photoresist and photo-crosslinkable polyethyle glycol may provide improved membrane support (also referred to herein as a “scaffold”). The various dimensions of the support, such as opening sizes, bar thickness, or scaffold thickness, can be optimized. For example, the scaffolds or SiN membrane may be patterned to match the well density and spacing of multi-well plates or other cell culture arrays. The scaffold materials may vary and may not be limited solely to photoresist. For example, the scaffold may be fabricated of PVDF, PTFE, cellulose, nylon, PES, or any plastic, metal, or other material that can be laser cut or otherwise formed into a supporting mesh scaffold to support the SiN membrane. Other examples of suitable scaffold materials include fluorinated polymers (e.g., highly fluorinated polymers) or fluorinated photoresists (e.g., highly fluorinated_photoresists.) Methods of making SiN membranes may be based on transfer of the nanoporous structure of a nanoporous silicon film (e.g., pnc-Si) or nanoporous silicon oxide film to a SiN film. Embodiments disclosed herein use a pore transfer process that uses pnc-Si or nanoporous silicon oxide film as a template for patterning SiN to have pores (also referred to as nanopores). Embodiments disclosed herein also use a process that lifts porous (also referred to as nanoporous) SiN membranes from the front surface of a Si wafer to avoid a through-wafer chemical etching process, which may be expensive and time consuming This may result in production of membranes with increased area and membranes that are more mechanically robust. For example, the membrane may have an area as large as a 150 mm Si wafer, which is approximately 177 cm.sup.2, an 200 mm Si wafer, or any glass or ceramic substrate that meets form factor and thermal requirements for a particular deposition, annealing, or liftoff process. The various steps disclosed herein may be performed on either a single wafer or batch of wafers.

In an embodiment, the method comprises: forming a nanoporous silicon film (e.g., pnc-Si film) or nanoporous silicon oxide film that is disposed on an SiN layer; etching said nanoporous silicon film (e.g., pnc-Si film) or nanoporous silicon oxide film such that pores in the SiN layer are formed during the etching. In another embodiment, the method further comprises the step of releasing the layer such that a free standing nanoporous SiN layer is formed. In an embodiment, the present disclosure provides a structure comprising a pnc-Si film as described herein disposed on a SiN film (a non-sacrificial film) as described herein.

The pnc-Si layer can be formed by methods known in the art. For example, the pnc-Si layer is formed by deposition of an amorphous silicon layer and subsequently depositing a silicon oxide layer on the amorphous silicon layer. The amorphous silicon layer and silicon oxide layer are heat treated under conditions such that a pnc-Si layer is formed. The silicon oxide layer may be a sacrificial layer that is removed after formation of the pnc-Si layer. In an embodiment, the pnc-Si layer is formed as described in U.S. Pat. No. 8,182,590, the disclosure of which is incorporated herein by reference.

In an embodiment, the pnc-Si mask is oxidized to form an SiO.sub.2 mask, e.g., during a thermal process carried out prior to the RIE transfer process. Some or all of the pnc-Si mask may be converted to the SiO.sub.2 mask during the oxidation, so some or none of the pnc-Si mask layer may remain. Depending on the source gas or gases used for the etching, this results in a SiO.sub.2 mask layer with greater etch selectivity. The oxidation also may reduce the pore size of thicker pnc-Si films because oxidation increases the volume by approximately 60% and constricts the pores.

The membranes may be produced on materials other than Si. For example, the membranes may be produced on stainless steel, Al.sub.2O.sub.3, SiO.sub.2, glass, or other materials known to those skilled in the art. Such materials may have certain surface roughness or temperature stability characteristics. For example, the surface roughness may be greater than a root means square (RMS) roughness of approximately 1 nm. However, this surface roughness may be limited based on degradation of the membrane quality for certain applications. Furthermore, these alternate materials may need to maintain structural integrity during pore formation because the membrane may achieve temperatures up to approximately 1000.degree. C. Certain materials, such as fused SiO.sub.2, Al.sub.20.sub.3, or other materials known to those skilled in the art, may be used to withstand the heating process. Fused SiO.sub.2 or Al.sub.20.sub.3 both may be transparent to most of the spectrum generated by the heat lamps during the annealing process to create nanopores. However, other materials, such as Mylar®, Teflon®, or Al may be used if higher temperatures are localized at the membrane.

The membranes may be produced on round or rectangular surfaces. Use of a rectangular surface may enable conveyor-style or roll-to-roll style production of the membranes. While particular membrane dimensions are disclosed, larger membranes on the order of greater than approximately 1 m.sup.2 may be possible using the methods disclosed herein. The structure of a nanoporous silicon film (e.g., pnc-Si film) or nanoporous silicon oxide film can be transferred to other thin films, such as SiN, SiO.sub.2, Al.sub.20.sub.3, high temperature oxides, single-crystal Si, or other materials, by using the a nanoporous silicon film (e.g., pnc-Si film) or nanoporous silicon oxide film as a mask during a reactive ion etching (RIE) process. RIE uses a chemically-reactive plasma to remove material and the chemistry of the RIE may vary depending on the thin film material. During this transfer, the open pores of the pnc-Si or silicon oxide allow incident ions to remove material from the SiN film while the nanocrystalline regions of the pnc-Si protect the SiN. Besides removing material from the SiN film, the RIE may also thin the pnc-Si or silicon oxide. The pnc-Si or silicon oxide may remain on the SiN or may be completely removed from the SiN during the RIE. For example, gases such as CF.sub.4, CHF.sub.3, SF.sub.6, and Ar, can be used during RIE. Additionally, gases such as O.sub.2 and H.sub.2 can be used in combination with the aforementioned gases during RIE

The pores in the SiN may correspond to the position of the pores in the pnc-Si. In an example, the pores are a near copy of each other.

Removing the pnc-Si layer may provide more consistency in the resulting SiN nanoporous film. For example, the residual mask may be non-uniform following the etch. Removing the residual mask may reveal a clean or uniform surface.

Lateral etch propagation may be affected by the interface between the oxide and nitride and/or Si. Thus, the type of these materials may be optimized. For example, SiO.sub.2 may be formed using TEOS, thermal processes, or sputter deposition at various thicknesses. The SiO.sub.2 may have a thickness between approximately 25 nm and 250 nm. The thickness of the sacrificial oxide may vary between approximately 25 nm and 150 nm.

Use of RIE allows a range of pore sizes and porosities to be formed in SiN films. The pore size and/or porosity of the resulting SiN film can be larger, smaller, or the same as that of the nanoporous silicon or silicon oxide mask.

Some factors that affect the pore transfer process and resulting pore geometry include the etch time, the chamber pressure, the source gases used, and the ratio of the various source gases used. Shorter etch times may lead to pore sizes that are comparable or less than that of the template material, such as that of the pnc-Si. Shorter etch times also may leave the pnc-Si or silicon oxide as a nanoporous cap on the SiN. In the case of pnc-Si, this cap may be used as a hydrophilic glass-like surface. Longer etch times may lead to pore side-wall erosion and, consequently, larger pore sizes and higher porosity in the SiN than the pnc-Si or silicon oxide. Increases in chamber pressure may decrease anisotropy and may result in larger pore sizes and porosity. Some source gases affect Si (or silicon oxide) differently from SiN. For example, CF.sub.4 etches Si faster than SiN while CHE.sub.3 reduces the etch rate of Si compared to SiN. This may be because the hydrogen in CHF.sub.3 increases the etch resistance of Si, but does not affect the etch rate of SiN. In contrast. Ar etches materials using a physical mechanism independent of the material being etched, which results in anisotropic etching. Various ratios of the source gases may be optimized to obtain particular results. Additional gases also may be used. For example, O.sub.2 may be used as an etchant to remove any fluoropolymers that form from the CF.sub.4 and CHF.sub.3 used for etching.

In an embodiment. XeF.sub.2 gas is used to remove the residual pnc-Si mask from the SiN, XeF.sub.2 has a 2000:1 etch selectivity between Si and SiO.sub.2 or SiN. Thus, less SiN is etched during this process, which may increase the overall strength of the membrane. The pnc-Si or silicon oxide mask can be removed by the etch process. In an embodiment, the pnc-Si or silicon oxide mask is completely removed during the etch process. In another embodiment, at least a portion of the pnc-Si or silicon oxide mask remains after the each process. In the case of a pnc-Si mask, the remaining pnc-Si can form a hydrophilic cap on the nanoporous SiN layer. The cap may help the SiN surface become more hydrophilic. This cap also may provide better wetting properties for the SiN membrane or increase overall permeance. SiN may be hydrophobic, which may impede water from passing through the pores. Rendering the SiN hydrophilic through the presence of this cap may reduce or eliminate this 20 characteristic of some SiN membranes.

The nanoporous SiN membrane also may be released from the surface of a Si wafer by supporting the SiN membrane with a polymer-based scaffold and chemically etching an adhesive SiO.sub.2 that bonds the SiN membrane to the Si wafer. This process can be referred to as a “lift-off” process. This polymer scaffold may provide more flexibility to the membrane sheet than SiN scaffolds. The SiN membrane and scaffold may be configured to release together so that the SiN membrane and scaffold remain intact during processing.

In an embodiment, a photosensitive polymer such as photoresist is used to pattern a scaffold on the membrane top side. This may create, in an example, an 80% porous scaffold. An etch is performed through the pores of the membrane using a BOE to preferentially etch the SiO.sub.2 at a >200:1 ratio compared to the SiN membrane. Thus, the SiO.sub.2 etches significantly faster than SiN whereas pnc-Si is not etched by the BOE. In another embodiment, vapor phase HF is used to chemically etch the SiO.sub.2 and release the SiN membrane. The SiN membrane can be released using other methods. The layer under the SiN membrane may be Si or the Si water and an XeF.sub.2 etch may be used to remove the Si in contact with the SiN. This would release the membrane in a dry etch process, which may provide a yield increase compared to some wet etch processes. In an example, a layer of polysilicon is disposed between the SiN membrane and a SiO.sub.2 layer. The polysilicon layer is dissolved by the XeF.sub.2 and the SiN membrane floats off the SiO.sub.2 layer.

The concentration of BOE or vapor phase HF and the etch time can be optimized to remove the sacrificial oxide without compromising the SiN membrane. BOE has a high etch selectivity for SiO.sub.2 compared to SiN. This selectivity may be approximately >200:1. Prolonged exposure to BOE may result in thinning and pore enlargement of Si or SiN membranes because BOE will eventually etch SiN during this prolonged exposure. Etching SiN 10 by 10 mm or more may enlarge and merge pores to the point that membrane strength is affected, though other factors also may play a role in the membrane strength.

An inorganic scaffold instead of a polymeric scaffold may be used in another alternate embodiment. Such inorganic scaffolds can be used in aggressive solvent systems or at temperatures greater than, for example, approximately 300.degree. C. Use of such inorganic scaffolds may enable these membranes to be used in the environments common to, for example, solid oxide fuel cells, nanopartiele production, hydrogen production, heterogeneous catalysis, or emissions control. Examples of inorganic scaffold materials include SiO.sub.2, SiN. Si, SiC, Al.sub.20.sub.3, and other materials known to those skilled in the art. Inorganic scaffolds May 3.5 be formed using methods such as, for example, soft lithography, LPCVD, or plasma-enhanced chemical vapor deposition (PECVD). Soft lithography may involve use of “green” state ceramic precursors and may create a scaffold pattern directly followed by drying and heat treatment (e.g., calcining). Certain types of chemical vapor deposition (CVD) may be followed by lithographic treatments to create the desired scaffold pattern.

In an embodiment, an oxide may be deposited or grown on the nanoporous SiN membrane to improve cell adhesion and wettability of the membrane. Etching during production of the SiN membrane may remove any capping pnc-Si, so the presence of this oxide may promote cell attachment to the SiN membrane. Alternatively, an extracellular matrix coating may be used to promote cell attachment to the SiN membrane instead of the oxide layer.

The properties and characteristics of the SiN membrane, including pore size, may vary as disclosed herein with the potential application. In an embodiment, the properties of the SiN, such as stress, thickness, or Si content, can be tuned or altered during manufacturing to suit a particular application. For example, strength of the SiN membrane may be increased by increasing the thickness.

Capturing and retaining extracellular vesicles on a nanoporous silicon nitride membrane provides an outstanding platform to conduct analysis of the presence of biomarkers of interest on the captured extracellular vesicles. As will be further described herein, an assay is used that may comprise various reagents such as a fluorochrome-antibody combination which is added to a fluid that contains extracellular vesicles. Certain reagents will attach to a biomarker of interest on the extracellular vesicle. This labelled extracellular vesicle is then captured by the nanoporous silicon nitride membrane and is in turn excited by a light source of a frequency sufficient to excite the fluorochrome-antibody combination, thus identifying the presence and quantity of the biomarker of interest. The nanoporous silicon nitride membrane acts as a capture and imaging scaffold, with the optically transparent properties of the nanoporous silicon nitride membrane providing an excellent platform for microscopy and other optical analysis techniques.

In using the nanoporous silicon nitride membrane, a biofluid containing extracellular vesicles and in some embodiments a fluorochrome-antibody combination is slowly passed over the nanoporous silicon nitride membrane under conditions of slight negative transmembrane pressure. This configuration permits the diffusion of extracellular vesicles toward the nanoporous membrane, such that the extracellular vesicles are captured in the pores of the membrane. While maintaining a negative transmembrane pressure, the extracellular vesicles can be retained in the pores while the fluid component of the biofluid is swept and cleared away, thus removing unwanted constituents from the biofluid. While maintaining transmembrane pressure, the captured extracellular vesicles can be washed in a clean solution to increase their purity. In some embodiments of the present invention, the transmembrane pressure can be released or reversed to slightly positive and the isolated extracellular vesicles are eluted off the membrane in a bolas of clean solution.

Once captured, the extracellular vesicles or other target cells are imaged using microscopy or other techniques to look for biomarkers that fluoresce when excited with a given wavelength of light. These fluorescing biomarkers are the result of the addition of an antibody-flourochrome reagent that has bound with the biomarker of interest on the extracellular vesicle.

The detection of biomarkers has broad applicability, including, but not limited to, the detection of disease and prediction of response to a therapy. Detection may include the detection of two or more biomarkers on a single extracellular vesicle. For example, the detection of immune checkpoint proteins is fundamentally important to many cancer treatments such as immunotherapies where it becomes important to assess antitumor immune status. In immune therapies, the activation of inhibitory checkpoint proteins in response to antitumor therapy undercuts therapeutic efficacy. The present invention provides a way to sample over time for the induction of checkpoint proteins to know if a checkpoint blockade is necessary. The present invention provides for testing of checkpoint inhibitors without tumor body sampling, and allows for the sampling over time once therapy is initiated and/or the tumor is removed.

A method for the detection of immune checkpoint proteins in accordance with the present invention comprises the steps of providing a biofluid, passing the biofluid over a nanoporous membrane wherein the nanoporous membrane comprises a plurality of pores, capturing with the nanoporous membrane extracellular vesicles contained within the biofluid, adding an antibody-fluorochrome combination to the extracellular vesicles, exciting the captured extracellular vesicles with a wavelength of light sufficient to fluoresce the antibody-fluorochrome combination, and identifying the excited captured extracellular vesicles. Alternatively, biomarker labeling may occur prior to extracellular vesicle capture.

The method may also include counting the excited captured extracellular vesicles where counting may be performed with a machine vision system and a counting program.

The physical sieving mechanism described herein where the extracellular vesicles are captured on the pores of the nanoporous silicon nitride membrane by diffusion into the slight transmembrane pressure environment of the porous membrane, in the context of a tangential flow configuration of the present invention, seems to depend on an excess of pores relative to the number of extracellular vesicles in the biofluid. Thus, a large pore-to-extracellular vesicle ratio is required for the isolation mechanism of the present invention and will likely only work with highly permeable membranes with a large density of pores (e.g., ˜:10⁷ pores per mm²).

The tangential flow configuration described herein results in the apparent removal of the unwanted but highly abundant species within most biofluids, with little residual contamination. For example, the high protein content of plasma can be removed from captured extracellular vesicles so that a highly pure extracellular vesicle preparation is realized.

In some embodiments of the present invention, the nanoporous silicon nitride membrane is chemically functionalized to add chemical selectivity. Chemical functionalization may include the use of amphiphilic molecules with proteins and antibodies that attach to the surface of the membrane such that the antibodies then interact with and capture biomarkers or other analytes of interest. Such chemical selectivity allows for the use of pores in the nanoporous silicon nitride membrane that are larger than the target cell where the target cells are captured by chemical binding when they come in close proximity to the surface of the membrane. Such chemical capture expands the analytical capabilities of the present invention by improving the capture rate of target cells and also reducing the possibility of the nanoporous silicon nitride membrane to become clogged or otherwise fouled.

For a more thorough understanding of the present invention and the various embodiments described and envisioned herein, reference is now made to the Figures.

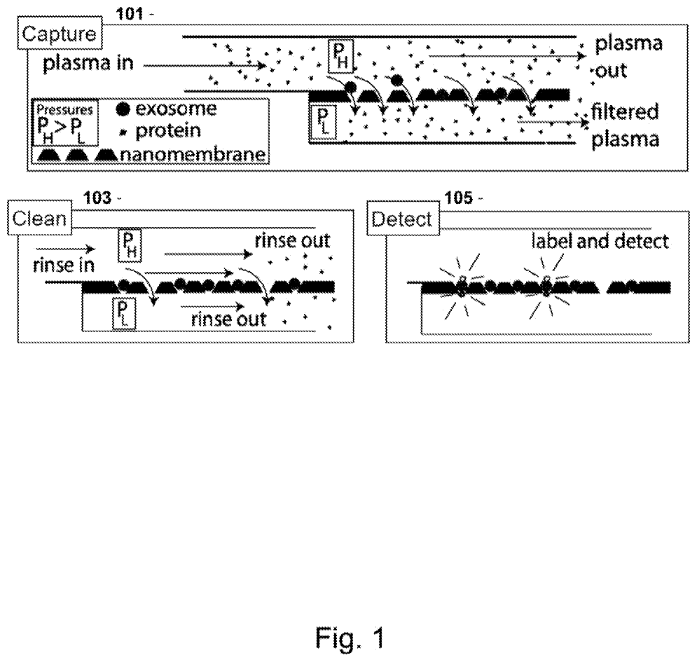

FIG. 1 depicts capture of exosomes and subsequent biomarker detection on a tangential flow device of the present invention. While tangential flow is described herein as an example, other flow configurations may also be employed with the present invention. In step 101 (Capture), exosomes are captured on a nanoporous silicon nitride membrane.

The vector labeled “plasma in” illustrates tangential flow across a nanoporous silicon nitride (NPN) membrane where a pressure gradient exists, providing a slightly lower pressure below the membrane than above the membrane, which pulls extracellular vesicles such as exosomes into the pores of the NPN membrane as protein is cleared. As labeled in FIG. 1, the extracellular vesicles are diagrammatically depicted as shaded circles and protein is diagrammatically depicted as a distorted asterisk of sorts. Such a membrane is described, for example, in United States Patent application publication 2016/0199787 A1 to Striemer et al. and entitled Nanoporous Silicon Nitride Membranes, And Methods For Making And Using Such Membranes, the entire disclosure of which is incorporated herein by reference. Other membranes, devices and methods applicable to the present invention and the various embodiments described, depicted and envisioned herein are disclosed in U.S. Pat. Nos. 8,518,276 and 8,501,668, the entire disclosures of which are incorporated herein by reference in their entirety.

For exosome capture in the tangential flow device of the present invention, in a preferred embodiment, transmembrane pressure in operation will be 1 pascal-1 atmosphere. Flow velocity will be 10 μm/sec.-10 cm./sec. Channel length will be 1 mm.-1 m, along the principal direction of flow. A large channel size may be used, for example in a large industrial size operation. Roll to roll processing, for example, could be used to create sheets of nanoporous silicon nitride (NPN). Channel height will be 100 nm.-1 mm. Pore diameter will be 20 nm.-120 nm., or in some embodiments of the present invention, 20 nm.-80 nm.

In step 103 (Cleaning), protein contaminants are removed by way of a rinsing process as depicted in FIG. 1. Once the extracellular vesicles are extracted from the plasma, a buffer solution is passed through the system to clear protein contaminants, leaving behind extracellular vesicles entrapped or otherwise captured in the nanoporous silicon nitride (NPN) membrane.

Once the extracellular vesicles are captured, in step 105 (Detect) an antibody-fluorochrome reagent is added to the captured extracellular vesicles (labeling). An appropriate wavelength of light excites the labelled extracellular vesicles where they are imaged and counted by way of microscopy and either manual or an automated (machine vision) system. Microscopy may include confocal microscopy, standard epifluorescent microscopy, high resolution microscopy, and the like. Counting of fluorescing biomarkers may be done manually, or by way of a counting program in a machine vision or optical analysis environment. Digital assays employ image processing techniques to identify type and quantity of analyte.

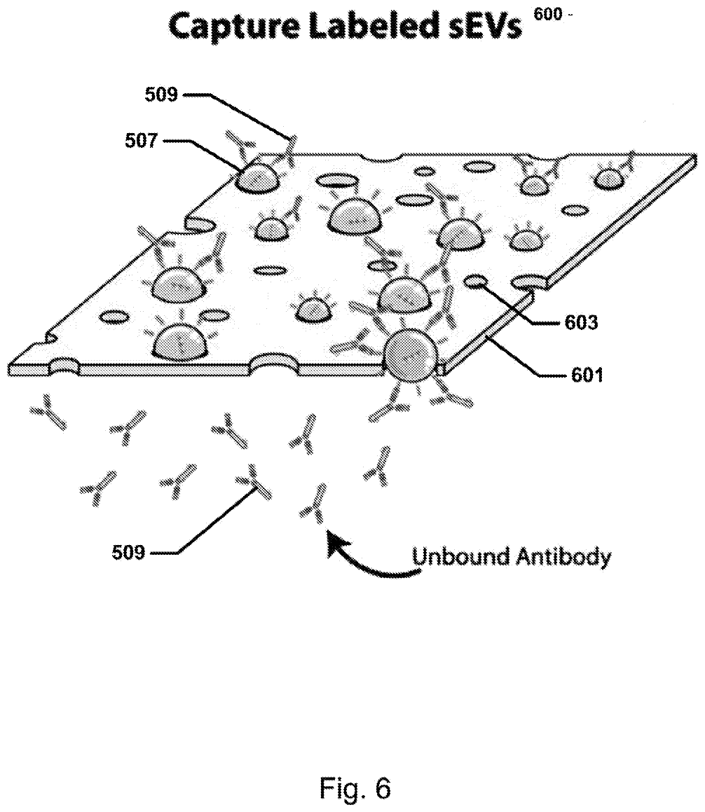

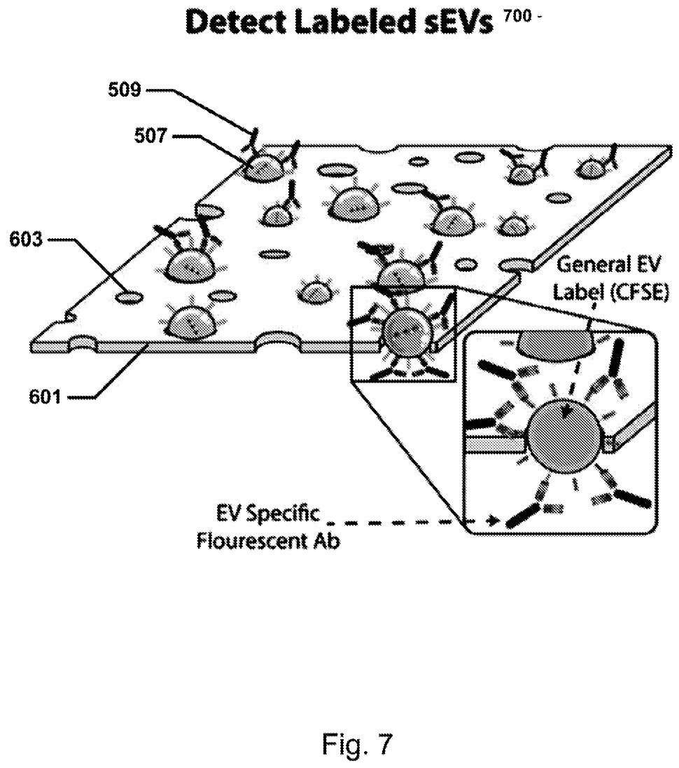

As will be later described by way of FIGS. 6-7, labeling of the extracellular vesicles in solution by way of an antibody-fluorochrome reagent may occur before the extracellular vesicles are captured by the nanoporous silicon nitride membrane.

Various antibody-fluorochrome reagents may be used in accordance with the present invention. In some embodiments of the present invention, quantum dots may be used instead of, or in addition to, fluorochromes.

For the biomarkers and functional assays described herein, multiple markers (or assays of function) can be used. These assays can have multiplexed extracellular vesicle (EV) labeling or functional assays performed simultaneously or in parallel or utilizing sequential detection procedures. This includes processes wherein individual markers (or functional assays) from within and between the listed groups below can be performed to permit a range of assays including quantification of the number, quantity of biomarker, activity level of functional targets, and co-localization of biomarkers and other functional characteristics of extracellular vesicles (EVs).

### Extracellular Vesicle Markers (EV, Including but not Limited to Small EV [Exosome] and Medium and Large BVs [Microvesicles]):

These markers include Tetraspanins (CD63. CD9, CD81), HSPA8, ALIX, ACTB, MSN, RAP1B and HSP90AB1 for EVs and Annexin Al specifically for microvesicles.

These EV markers can be combined for detection with the markers below to assess the presence of biomarkers and/or function in EVs.

### Cancer Markers:

Pan-cancer protein EV markers:

Include: versican (VCAN), tenascin C [TNC], thrombospondin 2 (THBS2).

Cancer EV protein markers for a multiple of cancers.

Include: septin 9 (SEPTIN9), basigin (BSG), fibulin 2 (FBLN2), four and a half LIM domains 2 (FHL2), inosine triphosphatase (ITPA), galectin-9 (LGALS9), splicing factor 3b subunit 3 (SF3B3), and calcium/calmodulin dependent serine protein kinase (CASK), cathepsin B (CTSB), all-trans-retinol dehy-drogenase [NAD (+)] ADH1B/alcohol dehydrogenase 1B [ADH1B], adenosylhomocysteinase [AHCY], and phosphoglycerate kinase 1 [PGK1], brain-specific angiogenesis inhibitor 1-associ-ated protein 2-like protein 1 (BAIAP2L1), alkaline phosphatase, tissue-nonspecific isozyme (ALPL), receptor-type tyrosine-protein phosphatase eta (PTPRJ), high-affinity immunoglobulin epsilon receptor subunit gamma (FCER1G), and cell surface hyaluronidase (TMEM2), leucine-rich repeat-containing protein 26 (LRRC26), ATP-dependent translocase ABCB1 (ABCB1), bile salt export pump (ABCB11), adhesion G-protein coupled receptor G6 (ADGRG6), desmocollin-1 (DSC1), desmoglein-1 (DSG1), keratin, type II cuticular Hb1 (KRT81), and plasminogen-like protein B (PLGLB1).

Serum cancer protein EV markers for pancreatic or colorectal cancer:

Include: immunoglobulin lambda constant 2 keratin 17, immunoglobulin heavy constant gamma 1, keratin 6B, ferritin light chain radixin, cofilin 1, protease, serine 1, tubulin alpha 1c. ADAM metallopeptidase with thrombospondin type 1 motif 13, immunoglobulin kappa variable 6D-21, tyrosine 3-monooxygenase/tryptophan S-monooxygenase activation protein theta, POTE ankyrin domain family member I, POTE ankyrin domain family member F von Willebrand factor, actin gamma 1, immunoglobulin lambda variable 3-27 immunoglobulin kappa variable 1D-12 coagulation factor XI, complement C1r subcomponent like attractin, butyrylcholinesterase immunoglobulin heavy variable 3-35 immunoglobulin kappa variable 1-17, C1q and TNF related 3 immunoglobulin heavy variable 3-20, immunoglobulin heavy variable 3/OR15-7 collectin subfamily member 11 immunoglobulin, heavy constant delta immunoglobulin kappa variable 3D-11 immunoglobulin heavy variable, 3/OR16-10 immunoglobulin kappa variable 2D-24 immunoglobulin kappa variable 2-40, immunoglobulin kappa variable 1-27 immunoglobulin heavy variable 3/OR16-9 immunoglobulin, lambda variable 5-45 immunoglobulin heavy variable 3/OR16-13, immunoglobulin heavy variable 1-46, immunoglobulin heavy variable 4-39, immunoglobulin heavy variable 3-11, immunoglobulin lambda constant 3, immunoglobulin kappa variable 1-6, paraoxonase 3, immunoglobulin heavy variable 3-21, immunoglobulin heavy variable 7-4-1, immunoglobulin kappa variable 2D-30, immunoglobulin lambda constant 6.

### Cancer Subtype Specific Markers:

Include (table from Shen, M., Di, K., He, H. et al. Progress in exosome associated tumor markers and their detection methods. Mol Biomed 1, 3 (2020). https://doi.org/10.1186/s43556-020-00002-3).

| | |
|---|---|
| colorectal cancer | Copine III |
| | CD147 |
| pancreatic ductul | GPC-1 |
| adenocarcinoma | |
| Gastric cancer | HER-2/neu, EMMPRIN, MAGE-1, C-MET |
| | TRIM3 |
| Prostate cancer | PSA |
| | ephrinA2 |
| | survivin |
| melanoma | (phospho)Met |
| | caveolin-1 |
| Renal cell carcinoma | MMP-9, DKP4, EMMPRIN, PODXL |
| (RCC) | |
| non-small-cell lung | EGFR, KRAS, claudins and RAB-family |
| carcinoma | proteins CD151, CD171 and tetraspanin 8 |

### Protein EV Markers of Tumor Prognosis:

Including: urine-derived EV Integrin alpha v beta 6 (ITGA3) and Integrin Subunit Beta 1 (ITGB1) and serum-derived Programmed death ligand 1 (PD-L1).

EV Markers Demonstrating the Need for, or Predicting Response to Therapy:

Including: (table from Zhou B, Li Y, Wu F, Guo M, Xu J, Wang S, Tan Q, Ma P, Song S, Jin Y. Circulating extracellular vesicles are effective biomarkers for predicting response to cancer therapy. EBioMedicine.: 2021 May; 67: 103365. doi: 10.1016/j.ebiom.2021.103365, Epub 2021 May 7. PMID; 33971402; PMCID: PMC8121992),

Circulating EV-proteins in cancer therapeutic response.

| | | | | | Potential |
|---|---|---|---|---|---|
| Cancer | Biological | Therapy | Biomarkers | Therapeutic | application in |
| Types | Fluids | Involved | in EVs | Changes | cancer patients |
| NSCLC | Blood | Immunotherapy | 10 proteins | Decreased | Activation of |
| | | | | | immune |
| | | | | | response |
| Prostate | Urine | Hormonal | PSA | Decreased | Monitoring |
| cancer | | therapy | PSMA | | treatment- |
| | | | 5T4 | | related |
| | | | | | responses |
| Prostate | Serum | Chemotherapy | MDR-1 | NA | Predicting |
| cancer | | | MDR-3 | | therapeutic |
| | | | Endophilin- | | resistance |
| | | | A2 PABP4 | | |
| Prostate | Serum | Chemotherapy | P- | Increased | Predicting |
| cancer | | | glycoprotein | | therapy |
| | | | | | resistance |
| Breast | plasma | Chemotherapy | TRPC5 | Increased | Predicting |
| cancer | | | | | chemoresistance |
| Melanoma | Plasma | Immunotherapy | PD-L1 | Increased | Predicting |
| | | | | | beneficial |
| | | | | | treatment |
| | | | | | responses |
| Melanoma | Plasma | Immunotherapy | ΔExoPD-L1 | Decreased | Predicting |
| | | | | | responsive |
| | | | | | therapy |
| Melanoma | Serum | Immunotherapy | PD-1 | Increased | Predicting |
| | | | CD28 | | beneficial |
| | | | | | treatment |
| | | | | | responses |
| Melanoma | Serum | Targeted | MCSP | NA | Monitoring |
| | | therapy | MCAM | | treatment |
| | | | ErbB3 | | responses |
| | | | LNGFR | | |
| GBM | Plasma | Chemotherapy | CD63 | Decreased | Monitoring and |
| | | | EGFR | | predicting |
| | | | EGFRvIII | | therapy |
| | | | | | effectiveness |
| GBM | Plasma | Surgical | 11 proteins | Increased | Monitoring |
| | | resection | | | treatment |
| | | | | | responses and |
| | | | | | predicting |
| | | | | | relapse |
| HNSCC | Plasma | Chemoradiation | 119 | NA | Predicting and |
| | | therapy | proteins in | | assessing |
| | | | non- | | treatment |
| | | | responders; | | responses |
| | | | 39 proteins | | |
| | | | in complete | | |
| | | | responders | | |

### EV Immune Checkpoint Proteins:

Including the inhibitory checkpoint proteins: Programmed death ligand 1 (PD-L1), Cytotoxic T-Lymphocyte Associated Protein 4 (CTLA-4), Programmed cell death 1 receptor (PD-1), Adenosine A2A receptor (A2AR), B7-H3, B7-H4, B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), V-domain Ig suppressor of T cell activation (VISTA) and stimulating checkpoint proteins: CD27, CD28, CD40, CD122, CD137, OX40. Glucocorticoid-Induced TNFR family Related gene (GITR), Inducible T-cell costimulatory (ICOS).

### Tumor Stage and Grade, Invasive and Metastatic EV Protein Markers:

Include CD44, Wnt Family Member SA (WNT5a). Transforming Growth Factor Beta Induced (TGFB1), Serpin Family E Member 1 (SERPINE1), and Growth/differentiation factor-15 (GDF-15) for tumor subtype and behavior and integrins α6β4, α6β1 and αvβ5 for organ specific metastasis.

EV tumor microenvironment protein markers including those that assess signals that support or repress the antitumor immune response as well as those that support metastasis:

### Markers Demonstrating the Cell of Origin:

Immune cell markers:
T, B and NK cells and subtypes:
Memory and effector, tissue resident, naïve.

### Innate Immune Cell Types:

Macrophages, neutrophils, monocytes, neutrophils, basophils, eosinophils, red blood cells and stem cells and precursors from which they originate.

### Immune Cell Function:

Including identification and functional characterization of proteins for antigen presentation and antigen recognition, co-stimulatory and inhibitory receptors.

Organs, Tissues and Cell Subtypes there in:

Including: Renal, hepatic, pulmonary, gastrointestinal, pancreatic, splenic, lymph nodes and lymphatic, peripheral and central nervous system, bladder, muscle, tendon, ligament, bone, cartilage, bone marrow and blood, fat, skin and subdermal tissue, heart and vascular.

### Markers Elucidating Functional States of the Cell of Origin:

Including protein, RNA and DNA that indicates the cell of origin is in a stable or transient state of: senescence; activation; anergy; proliferation; cell stress; invasiveness; activated, repressed by, or mediating inflammation; is derived from cells modulated by cell intrinsic or cell extrinsic pathologic states including disease states due to genetic, environmental, aging, hypoxic, degenerative, infectious and inflammatory causes.

### Covalent Modifications of Proteins, RNA and DNA:

Including modification of proteins, RNA and DNA including phosphorylation, acetylation, methylation, myristoylation. ADP-ribosylation, farnesylation, ubiquitination, γ-Carboxylation, and sulfation and the presence of the proteins that add and remove these modifications.

### Assessing the Bioactivity of EV Components:

Monitoring the presence of active proteins including enzymes, channels, receptors. ligands, signal transduction machinery.

### Screening Therapeutic Molecules or Reagents:

Testing the biological activity of potential therapeutics including drug/reagent binding kinetics, uptake and export, ability to modulate targets in, or function of EVs.

### Extracellular Vesicle Contents:

### RNA and DNA:

Small RNA, miRNA, t and Y RNA, mRNA, long noncoding RNA.

### Proteins Including Cytokines, Chemokines, Growth Factors, Receptors and Ligands

It should be noted that pore size of the nanoporous membrane is a variable that can be tuned to accommodate a variety of analytes. Pore geometry is a variable in the capture of the analyte, both size and spacing. Spacing of the pores is related to the resolution of the microscope used in the analysis. For example, counting of the analytes is improved when the pores are spaced apart, but this also reduces sample size.

In some embodiments of the present invention, various coatings and layers are applied to the nanoporons silicon nitride membrane. For example, very thin molecular layers with excellent hydrolytic stability may be employed. For example, a layer of 1-10 nanometer thickness. Such layers are designed so as not to occlude the pores or reduce permeability of the membrane. Such coatings provide enhanced surface interactions to assist in the capture of plasma components to supplement or otherwise interact with fluidic forces in the tangential flow device of the present invention.

An example of such a layer is that which is produced by functional carbene precursors to form uniform, Si—C and C—C attached monolayers on silicon, silicon nitride, and inert organic polymers under mild vacuum conditions. By utilizing meta-stable carbene species generated under mild UV-light illumination, the activation barrier for the Si—C and C—C bond formation is reduced and the variety of functional groups and surfaces that can be modified through surface-grafting reactions is expanded.

Ultrathin nanoporous silicon nitride (NPN) membranes can be functionalized with stable and functional organic molecules via carbene insertion chemistry. One example of a suitable organic coating for NPN is a thin, inert polymer layer that serves as the carbene attachment layer, and a stable polyethylene glycol (PEG) terminated monolayer that is linked to the polymer via non-hydrolytic C—C bonds generated by the vapor-phase carbene insertion. Such modifications to NPN provide the desired organic functionalities without significantly impacting pore size distribution or transport properties.

Coatings and monolayers for a substrate such as nanoporons silicon nitride (NPN) that may be employed with the present invention are described in U.S. patent application Ser. No. 15/130,208 to A. Shestopalov, L. Xunzhi and J. L. McGrath filed on Apr. 15, 2016 and entitled “Methods for Depositing a Monolayer on a Substrate Field”, the entire disclosure of which is incorporated herein by reference in it's entirety.

By defining surface chemistries, species capture from plasma can be controlled and selective capture of plasma components can be realized. Different chemical handles can be used to functionalize NPN membranes. Mixtures of different chemical handles can be used to further modulate the levels of adsorption of the plasma components and also to enhance adsorption selectivity. These chemical handles can be used in combination with different tangential flow regimes and membrane pore sizes to enhance specificity and selectivity of the 20 membrane-plasma component interactions.

In the device of the present invention, there are three distinctive interfaces between the nanoporous silicon nitride (NPN) and blood plasma or other biofluid that act as non-binding, adsorbing, or selective surfaces for the selective removal of components such as extracellular vesicles. Individually these defined surfaces will (1) non-specifically limit adsorption of biomolecules from the plasma solution by creating water-like solvating environments near the interfaces (e.g . . . polyethylene glycol molecules or zwitterionic species), (2) non-selectively enhance adsorption of various biomolecules through ionic interactions and H-bonding (e.g., aminated interfaces), and (3) selectively bind serum components via specific biomolecular interaction (e.g., antigen-antibody interactions or specific H-bonding). Therefore, by creating homogeneously mixed monolayers that contain different ratios of non-binding, adsorbing, and selective species, capture selectivity can be established by the defined flow parameters and can further be enhanced by controlling the chemical composition of the membrane walls.

Defined surface chemistries may include, for example, antibodies that capture extracellular vesicles. Capture of extracellular vesicles by affinity using antibodies may include tangential flow arrangements such as those described and envisioned herein. In addition, antibodies may be combined with other defined surface chemistries for specific applications. There are also antibodies that are specific to extracellular vesicles. For example, CD63, CD9, CD81 and Hsp70 all have affinity to exosomes. The present invention and the various embodiments described, depicted and envisioned herein includes generically the employment of antibodies in general to capture, move, sort, retain, and otherwise process extracellular vesicles.

It is further stated that the various techniques, devices, methods and apparati described herein are also suitable for the capture of other cells or cell components that may contain biomarkers and where the devices and methods described herein are suitable for such biomarker capture and detection.

The carbenylation approach can be used as a simple, robust and universal method to functionalize nanoporous materials with diverse classes of organic and biological species. The inventors have demonstrated that carbenylated monolayers on Si, Ge, SiN, ITO and polymers can be modified with various organic and biological molecules—small molecules, PEG-oligomers, GFP proteins and others-via simple surface reactions, and that they exhibit excellent hydrolytic stability in water and aqueous buffers for up to 2 weeks of exposure.

To form functional monolayers on nanoporous silicon nitride (NPN), the membranes will first be modified with an inert aliphatic coating that serves as a passivating layer and as a carbene attachment interface. Subsequently, the NHS-diazirine carbene precursors will be used to deposit the NHS-terminated monolayers on the aliphatic coating through the thermodynamically and hydrolytically stable C—C bonds. Lastly, individual or mixed NH2-terminated molecules (non-binding, adsorbing, and selective) will be reacted with the NHS-terminated monolayer to modify the resulting membranes with the desired chemical functionalities.

Nanoporous silicon nitride membranes with 100-1,000 nanometer diameter pores are fabricated with patterning and etching methods. Specifically, 30 nanometer diameter pore membranes are fabricated using methods disclosed in PCT/US2014/1051316, the entire disclosure of which is incorporated herein by reference. The 30 nanometer pore size of nanoporous silicon nitride (NPN) membranes allows for the capture and retention of 30-100 nanometer extracellular vesicles such as exosomes, while passing contaminating species such as <30 nm proteins. The large number of pores within these membranes (˜1.7×10⁸ pores/mm² assuming 35 nm pores and 16% porosity) exceeds the number of exosomes in most biofluids by several orders of magnitude (assuming 10⁵ exosomes/mL for plasma). This exosome-to-NPN pore ratio suggests that nanoporous silicon nitride (NPN) membranes can capture nearly 100% of extracellular vesicles such as exosomes while leaving a large number of pores unoccupied to enable the removal of smaller contaminants.

Analytical techniques such as the creation of computational models for exosome capture can be used to determine the relationship between flow parameters and the capture of exosomes of various sizes. Computational models may be built with finite element analysis software that includes modeling of Brownian particles to the flow field. The models may, for example, include the hydraulic permeability of ultrathin membranes and assume a Newtonian fluid with the viscosity of plasma. In any resulting model, fluid streamlines in the top sample channel are expected to be parabolic with a slight permeation through the membrane into the lower chamber. The particles far from the channel will experience a large drag force tangential to the membrane while those very close to the membrane will experience drag toward the membrane from transmembrane convection and diminished tangential drag force. Exosomes entering this ‘capture layer’ will be pulled into the pore of the membrane and held there so long as there is transmembrane pressure.

A computational model may predict, for example, the height of the capture layer as a function of the flow parameters. It is expected that most well built computational models will indicate that the capture layer will be very small compared to the channel height.

$$δ ≪ H$$

Thus it is only through diffusive excursions from the bulk to the membrane that most exosomes will become trapped in the membrane pores, and we can expect a Peclet defined as

To be a key predictor of exosome capture. Note that because the diffusion coefficient and the drag forces imparted by the fluid on a particle are both dependent on the friction factor f both will be dependent on the particle size r, and the probability of capture is expected to be strongly dependent on particle size. Use of such modeling will allow one to prescribe flow settings that tune the capture process to exosomes (or micro vessels) of a particular size. Use of such a model will allow determination of application specific dimensions to ensure complete capture of target particles (such as exosomes) from a flowable material in a single pass across the membrane of the present invention. Input pressures and channel dimensions are two such parameters. A computational model can also be used to prescribe pressures during the recovery process if simple ‘backwashing’ proves problematic in a given application and configuration. As previously described herein, defined surface chemistries may also be employed with the membrane of the present invention for specific applications or to improve the retention of desired material by the membrane, reject non-desired material, or remove the retained desired material when certain conditions (such as a pressure change) are applied.

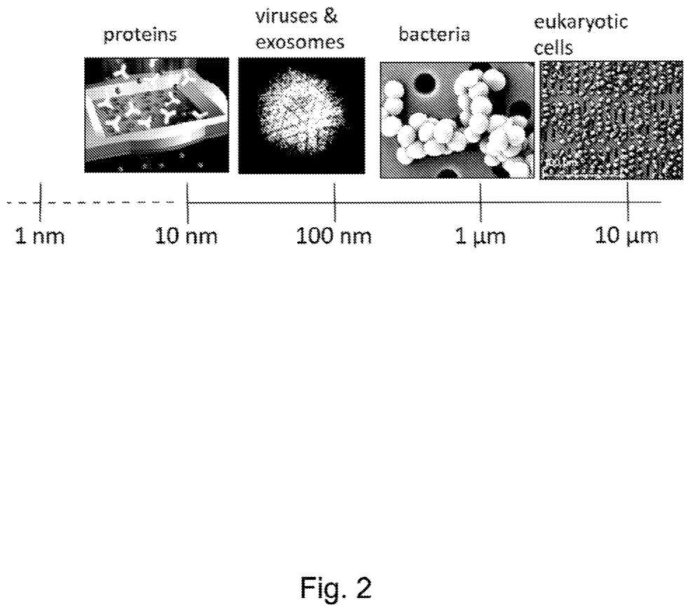

Turning now to FIG. 2, a chart depicting typical analyte sizes is shown. As previously described, pore geometry can be modified to accommodate capture of various analytes. In addition, chemical functionalization may be employed to aid in the capture and retention of analytes.

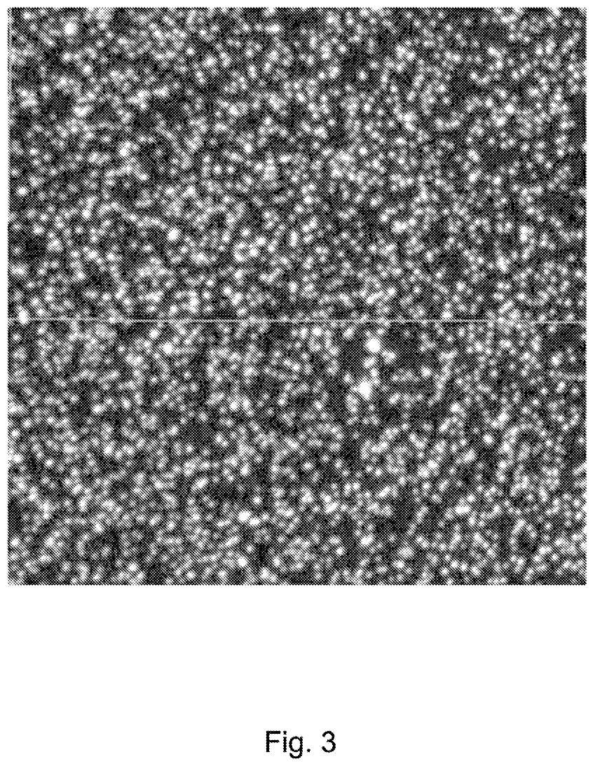

FIG. 3 illustrates the labeling of biomarkers on extracellular vesicles in accordance with the present invention. The brighter spots in the image represent fluorescing biomarkers. It should be noted that while FIG. 3 represents only intensity due to it's black and white representation, multiple assays containing multiple antibody-fluorochrome combinations may be employed to identify multiple biomarkers, each of which would fluoresce at a different wavelength, thus providing a multi-colored field of view that can be quantified by a digital assay such as a counting program with image processing.

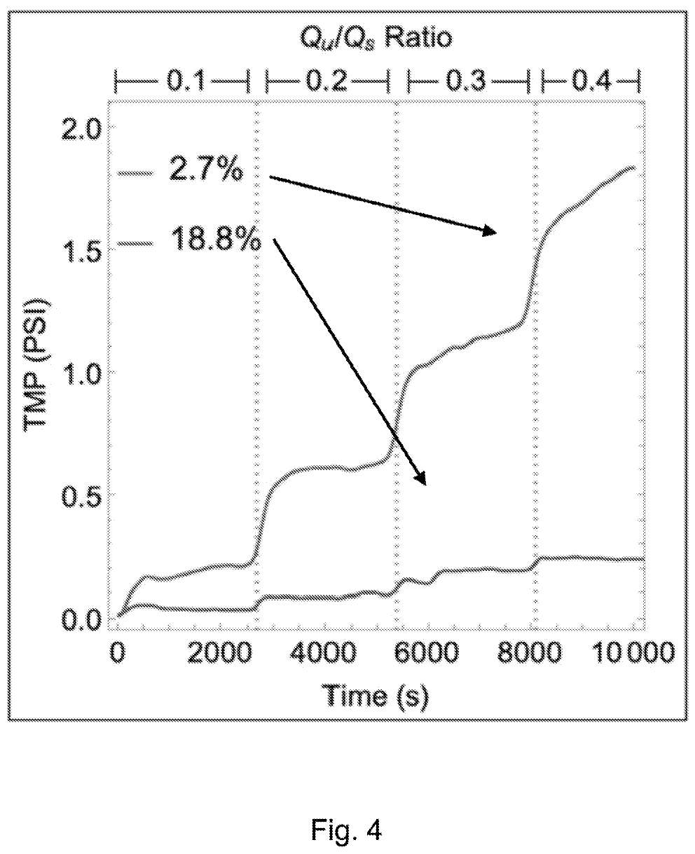

FIG. 4 is a graph depicting pressure with respect to time for a nanoporous membrane of the present invention where Qu represents flow through an exemplary membrane and Qs represents flow over the exemplary membrane.

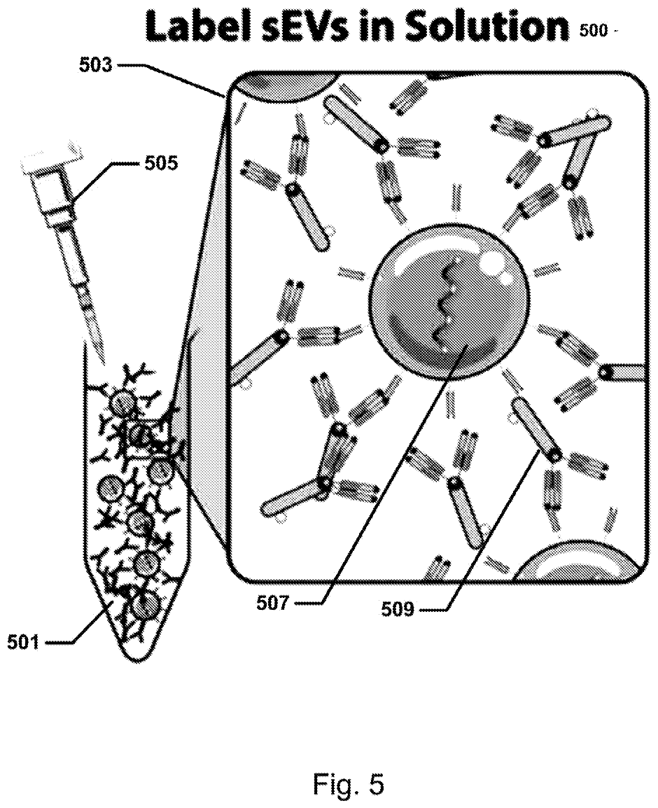

FIG. 5 illustrates the labeling of extracellular vesicles in solution where a reagent 505 comprising an antibody and a fluorochrome (or a light releasing marker such as quantum dots) are added to a solution 501 containing extracellular vesicles. As seen in the expanded view 503, the analyte 507 (perhaps a biomarker contained with the extracellular vesicle) receives or is otherwise bonded with an antibody 509 where the resulting structure fluoresces and can be viewed and counted with microscopy techniques such as those described herein.

FIG. 6 illustrates the capture of labeled extracellular vesicles in solution using a nanoporous membrane of the present invention. As previously described herein, a nanoporous silicon nitride membrane 603 retains an analyte 507 by way of retention in a pore 603. An antibody 509 attaches to the analyte 507 where the analyte can then be counted by way of optical techniques such as those described herein.

Lastly, FIG. 7 depicts detection of labeled extracellular vesicles using a fluorescent antibody combination. The attached antibody-fluorochrome 509 is retained by pores 603 within the nanoporous silicon nitride membrane 603 where the captured and labelled extracellular vesicles can be excited by the appropriate wavelength of light and then detected and counted using a digital assay technique such as those described previously herein.

It is, therefore, apparent that there has been provided, in accordance with the various objects of the present invention, a nanomembrane device and method for biomarker sampling.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, claims and drawings appended herein.

What is claimed is:

1. A device for the detection of immune checkpoint proteins, the device comprising:
a nanoporous membrane comprising a plurality of pores;
the nanoporous membrane configured to capture extracellular vesicles; and
an assay to determine the level of immune checkpoint proteins contained with captured extracellular vesicles.

2. The device of claim 1, wherein the assay comprises microscopy.

3. The device of claim 1, wherein the assay comprises an antibody.

4. The device of claim 1, wherein the assay comprises an antibody-fluorochrome combination.

5. The device of claim 1, wherein the assay comprises quantum dots.

6. The device of claim 1, wherein the nanoporous membrane is nanoporous silicon nitride.

7. The device of claim 1, wherein the density of pores of the nanoporous membrane is at least 10⁵ pores per square millimeter.

8. The device of claim 1, wherein the range of pore diameters in the nanoporous membrane is on the average between 20 nanometers and 120 nanometers.

9. The device of claim 1, wherein the device further comprises a machine vision system for imaging fluorescing immune checkpoint proteins.

10. The device of claim 9, wherein the machine vision system further comprises a counting program for counting the fluorescing immune checkpoint proteins.

11. A method for the detection of immune checkpoint proteins, the method comprising the steps of:
providing a biofluid;
passing the biofluid over a nanoporous membrane wherein the nanoporous membrane comprises a plurality of pores;
capturing with the nanoporous membrane extracellular vesicles contained within the biofluid;
adding an antibody-fluorochrome combination to the extracellular vesicles;
exciting the captured extracellular vesicles with a wavelength of light sufficient to fluoresce the antibody-fluorochrome combination; and
identifying the excited captured extracellular vesicles.

12. The method for the detection of immune checkpoint proteins as stated in claim 11, the method further comprising the step of:
counting the excited captured extracellular vesicles.

13. The method for the detection of immune checkpoint proteins as stated in claim 12, wherein counting is performed with a machine vision system and a counting program.

14. The method for the detection of immune checkpoint proteins as stated in claim 11, wherein the antibody-fluorochrome combination is indicative of immune checkpoint proteins.

15. The method for the detection of immune checkpoint proteins as stated in claim 11, wherein the antibody-fluorochrome combination comprises quantum dots.

16. A device for the detection of biomarkers, the device comprising:
a nanoporous membrane comprising a plurality of pores;
the nanoporous membrane configured to capture extracellular vesicles; and
an assay to determine the level of biomarkers contained with captured extracellular vesicles.

17. The device of claim 16, wherein the assay comprises an antibody-fluorochrome combination.

18. The device of claim 16, wherein the nanoporous membrane is nanoporous silicon nitride.

19. The device of claim 16, wherein the device further comprises a machine vision system for imaging biomarkers.

20. The device of claim 16, wherein the machine vision system further comprises a counting program for counting the biomarkers.
